(19) United States
(12) Patent Application Publication
(10) Pub. No.: US 2004/0247531 A1
(43) Pub. Date: Dec. 9, 2004

(54) Self-foaming, foam-like, after-foaming or foamable cosmetic or dermatological preparations containing waxes or lipids that are solid or semi-solid at room temperature

(75) Inventors: Heidi Riedel, Hamburg (DE); Andreas Bleckmann, Hamburg (DE); Ranier Kropke, Schenefeld (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/810,167

(22) Filed: Mar. 26, 2004

Related U.S. Application Data
(63) Continuation of application No. PCT/EP02/09156, filed on Aug. 16, 2002.

(30) Foreign Application Priority Data
Aug. 16, 2002 (DE) 101 47 820.8

(51) Int. Cl. A61K 9/00

(52) U.S. Cl. 424/47

(57) ABSTRACT

The invention is a self-foaming, foam-like, after-foaming or foamable cosmetic or dermatological preparation comprising at least one compound that is solid or semi-solid at room temperature selected from the group consisting of waxes and lipids. The cosmetic or dermatological preparation can be used for skincare or hair care and the at least one compound can be used to “boost” the foam of the preparation.

### CROSS-REFERENCE TO RELATED APPLICATIONS

This is a continuation application of PCT/EP02/09156, filed Aug. 16, 2002, which is incorporated herein by reference in its entirety, and also claims the benefit of German Priority Application No. 101 47 820.8, filed Sep. 27, 2001.

### FIELD OF THE INVENTION

The present invention relates to self-foaming, foam-like, after-foaming or foamable cosmetic and dermatological preparations, in particular skincare cosmetic and dermatological preparations.

### BACKGROUND OF THE INVENTION

Foams or foam-like preparations are a type of disperse system.

By far the most important and best known disperse system are emulsions. Emulsions are two- or multi-phase systems of two or more liquids which are insoluble or only slightly soluble in one another. The liquids (pure or as solutions) are present in an emulsion in a more or less fine distribution, which generally has only limited stability.

Foams are structures of gas-filled, spherical or polyhedral cells which are delimited by liquid, semi-liquid, high-viscosity or solid cell ribs. The cell ribs, connected via points of intersection, form a continuous framework. The foam lamellae stretch between the cell ribs (closed-cell foam). If the foam lamellae are disturbed or if they flow back into the cell rib at the end of foam formation, an open-cell foam is obtained. Foams are also thermodynamically unstable since a reduction in the surface area leads to the production of surface energy. The stability and thus the existence of a foam is thus dependent on to what extent it is possible to prevent its self-destruction.

Cosmetic foams are usually dispersed systems of liquids and gases, where the liquid represents the dispersant and the gas represents the dispersed substance. Foams of low-viscosity liquids are temporarily stabilized by surface-active substances (surfactants, foam stabilizers). Because of their large internal surface area, such surfactant foams have a high adsorption capacity, which is utilized, for example, in cleaning and washing operations. Accordingly, cosmetic foams are used, in particular, in the fields of cleansing, for example as shaving foam, and of hair care.

To generate foam, gas is bubbled into suitable liquids, or foam formation is achieved by vigorously beating, shaking, spraying or stirring the liquid in the gas atmosphere in question, provided that the liquids comprise suitable surfactants or other interface-active substances (“foam formers”), which, apart from interfacial activity, also have a certain film-forming ability.

Cosmetic foams have the advantage over other cosmetic preparations of permitting a fine distribution of active ingredients on the skin. However, cosmetic foams can generally only be achieved using particular surfactants, which, moreover, are often not well tolerated by the skin.

A disadvantage of the prior art is that such foams have only low stability, for which reason they usually collapse within approximately 24 hours. A requirement of cosmetic preparations, however, is that they have stability for years, as far as possible. This problem is generally taken into account by the fact that the consumer produces the actual foam himself just before use using a suitable spray system, for which purpose, for example, it is possible to use spray cans in which a liquefied pressurized gas serves as propellant gas. Upon opening the pressure valve, the propellant liquid mixture escapes through a fine nozzle, and the propellant evaporates, leaving behind a foam.

After-foaming cosmetic preparations are also known per se. They are firstly applied to the skin from an aerosol container in flowable form and, after a short delay, develop the actual foam only once they are on the skin under the effect of the after-foaming agent present, for example a shaving foam. After-foaming preparations are often in specific formulation forms, such as, for example, after-foaming shaving gels or the like.

However, the prior art does not include any sort of cosmetic or dermatological preparations which could be foamed as early as during the preparation and nevertheless have a sufficiently high stability in order to be packaged in the usual manner, stored and put onto the market.

### SUMMARY OF THE INVENTION

An object of the present invention was therefore to provide cosmetic or dermatological self-foaming or foam-like preparations which do not have the disadvantages of the prior art.

German laid-open specification DE 197 54 659 discloses that carbon dioxide is a suitable active ingredient for stabilizing or increasing the epidermal ceramide synthesis rate, which may serve to enhance the permeability barrier, reduce the transepidermal water loss and increase the relative skin moisture. To treat the skin, the CO₂ is, for example, dissolved in water, which is then used to rinse the skin. However, the prior art hitherto does not include any sort of cosmetic or dermatological bases in which a gaseous active ingredient could be incorporated in an adequate, i.e., effective, concentration.

It was thus a further object of the present invention to find cosmetic or dermatological bases into which effective amounts of gaseous active ingredients can be incorporated.

It was surprising and could not have been foreseen by the person skilled in the art that self-foaming, foam-forming, after-foaming or foamable cosmetic and dermatological preparations which comprise at least one wax or a lipid which is solid or semi-solid at room temperature overcome the disadvantages of the prior art.

### DETAILED DESCRIPTION OF THE PREFERRED EMBODIMENTS

For the purposes of the present invention, “self-foaming”, “foam-like”, “after-foaming” and “foamable” are understood as meaning that the gas bubbles are present in (any) distributed form in one (or more) liquid phase(s), where the preparations do not necessarily have to have the appearance of a foam in macroscopic terms. Cosmetic or dermatological preparations according to the invention (for the sake of simplicity also referred to as foams below) may, for example, be macroscopically visibly dispersed systems of gases dispersed in liquids. The foam character can, however, for example, be visible only under a (light) microscope. Moreover, foams according to the invention are—particularly when the gas bubbles are too small to be recognized under a light microscope—also recognizable from the sharp increase in volume of the system.

According to the prior art, self-foaming, foam-like, after-foaming or foamable cosmetic emulsions cannot be formulated or prepared industrially without the use of particular surfactants. This is true particularly for systems which are based on classic emulsifiers according to the prior art. Systems of this type, with the addition of propellant gas, developed exclusively aqueous-moist foams which rapidly broke following application.

As a result of the invention, a rich, compact cream foam is obtainable for the first time which is characterized by a long storage period, and by an extraordinarily high stability and a compact appearance.

As a result of the use of the waxes and/or lipids according to the invention, the introduction of gases is aided, and a stabilizing and significantly foam-boosting effect is achieved over a prolonged storage period, even at relatively high temperatures (e.g. 40° C.). It was particularly surprising here that the use of particular surfactants can be dispensed with. The introduction of gases is, surprisingly, extraordinarily increased compared with the prior art. Thus, for example, foam boosting with up to 100% increased gas volume can be achieved without using foaming agents customary according to the prior art, such as surfactants.

As a result of this, it is possible for the first time to stably generate formulations with an excellent, novel type of cosmetic activity and with an extraordinarily high gas volume (air and/or other gases such as oxygen, carbon dioxide, nitrogen, helium, argon etc.) over a long storage period at high temperatures. At the same time, the preparations according to the invention are characterized by above-average skincare and very good sensory properties.

The present invention thus further provides for the use of one or more waxes or a lipids which are solid or semi-solid at room temperature for boosting the foam of self-foaming, foam-like, after-foaming or foamable cosmetic and dermatological preparations.

For the purposes of the present invention, “foam boosting” is understood as meaning that the introduction of gases into the foams according to the invention is extraordinarily increased compared to the introduction into otherwise identical preparations which do not comprise waxes and/or lipids according to the invention. The foams according to the invention can, accordingly, take up a significantly greater gas volume than preparations which do not comprise waxes or lipids according to the invention.

Moreover, “foam boosting” means that the stability of the foamed preparations (the “foam stability”) compared with otherwise identical preparations which do not comprise waxes and/or lipids according to the invention is significantly improved, i.e. as a result of the use according to the invention, breaking of the foams is delayed.

In addition, for the purposes of the present invention, “foam boosting” is understood as meaning that the cosmetic properties of the foams according to the invention compared with preparations which do not comprise waxes and/or lipids according to the invention are also significantly improved: thus, the use according to the invention gives rich, solid foams (“foam creams”) which, despite their compactness and richness, can be spread easily and absorb rapidly.

The preparations according to the invention are entirely satisfactory preparations in every respect. It was particularly surprising that the foam-like preparations according to the invention are extraordinarily stable, even in cases of an unusually high gas volume. Accordingly, they are particularly suitable for use as bases for preparation forms having diverse intended uses. The preparations according to the invention have very good sensory properties, such as, for example, extensibility on the skin or the ability to be absorbed into the skin, and, moreover, are characterized by above-average skincare.

In accordance with the stipulations of the Deutsche Gesellschaft fur Fettwirtschaft [German Society for Fat Commerce] (Fette, Seifen, Anstrichmittel, 76, 135 [1974]), to designate the term “wax” usually involves taking into consideration the mechanical-physical properties of the waxes, that are relevant for their use, while the particular chemical composition is not taken into account for the definition.

“Wax”—like “resin”—is a collective term for a series of natural or synthetic substances which usually have the following properties: kneadable at 20° C., solid to brittly hard, coarsely to finely crystalline, transparent to opaque, but not glass-like, melt above 40° C. without decomposition, even a little above the melting point are relatively low-viscosity and not thread-drawing, have a considerable temperature-dependent consistency and solubility and can be polished under slight pressure. If, in borderline cases, a substance does not satisfy more than one of the abovementioned properties, then it is not a wax within the meaning of this definition. Waxes differ from similar synthetic or natural products (e.g. resins, plastic masses etc.) primarily by virtue of the fact that they convert to the molten, low-viscosity state usually between about 30 and 90° C., in exceptional cases also up to about 200° C. and are virtually free from ash-forming compounds.

Waxes for the purposes of the present invention are compounds which are characterized in that, together with the other oil components of the preparations according to the invention (such as, for example, polar, liquid compounds, UV filters and solvents thereof etc.), they form a mass which is spreadable or flowable at room temperature and which, at 20° C., have a viscosity of more than 500 mPa·s.

Advantageous waxes or lipids according to the invention are, for example, paraffin hydrocarbons, synthetic or semi-synthetic waxes or wax esters, and vegetable waxes and mixtures thereof with melting points or solidification points of from 25 to 125° C.

Advantageous waxes according to the invention are also those listed below:

| Class | Subgroup | Examples |
|---|---|---|
| Natural waxes | Vegetable | Candelilla wax, carnauba wax, japan |
| | waxes | wax, esparto grass wax, cork wax, |
| | | guaruma wax, rice germ oil wax, sugar |
| | | cane wax, ouricury wax, montan wax |
| | Animal waxes | Beeswax, shellac wax, spermaceti, |
| | | lanolin (wool wax), uropygial grease |
| | Mineral waxes | Ceresin, ozokerite (earth wax) |
| Chemically | Hard waxes | Montan ester waxes, sasol waxes, |
| modified waxes | | hydrogenated jojoba waxes |
| Synthetic | | Polyalkylene waxes, polyethylene |
| waxes | | glycol waxes |

Advantageous according to the invention are, for example, natural waxes of animal and vegetable origin, such as, for example, beeswax, Chinese wax, bumble-bee wax and other insect waxes, in particular those specified above.

Beeswax, for example, is an excretion product from the glands of honey bees, which the latter use to build honeycombs. Yellow (Cera flava), brown or red so-called crude wax is, for example, obtainable by melting the honeycombs freed from the honey by centrifugation, separating the melt from solid impurities, and allowing the resulting crude wax to solidify. The crude wax can be bleached completely white using oxidizing agents (Cera alba).

Beeswax consists of cerin, which is readily soluble in alcohol and is a mixture of cerotic acid CH₃(CH₂)₂₄COOH and melissic acid CH₃(CH₂)₂₈COOH, and of an ester mixture termed myricin consisting of about 70 esters of C₁₆- to C₃₆-acids and C₂₄- to C₃₋₆-alcohols. The essential constituents of beeswax are myricyl palmitate, myricyl cerotinate and paraffin.

Other insect waxes, such as, for example, bumble-bee wax, shellac wax or Chinese wax, are also essential mixtures of various esters. Chinese wax, for example, is deposited or produced in China and Japan from the wax scale louse (Coccus ceriferus) living on the Chinese ash, and the scale louse species Ceroplastes ceriferus and Ericerus pela. It is scraped from the trees and purified by remelting in boiling water. The main constituent of Chinese wax is the cerotic ester of ceryl alcohol.

Shellac wax is obtained from lac, the secretion of the female lac insects (Kerria lacca), which live in huge colonies (lac is derived from the Hindhi word “Lakh” for 100 000) on trees and shrubs in southern Asia (India, Burma, southern China). The shellac wax obtainable by solvent extraction contains, as essential constituents, myricyl alcohol, melissic acid and other wax alcohols and acids or esters thereof.

Plant waxes are also advantageous for the purposes of the present invention. Those preferably used are cuticular waxes of lower or higher plants, algae, lichens, mosses and fungi, such as, for example, candelilla wax, carnauba wax, Japan wax, esparto grass wax, cork wax, rice wax, sugar cane wax, fruit waxes, e.g. apple wax, flower waxes, leaf waxes from conifers, coffee wax, flax wax, sesame wax, jojoba oil and the like.

Candelilla waxes, for example, are brownish to yellowish brown, hard wax-like masses which are soluble in lipophilic solvents. Candelilla wax contains odd-number aliphatic hydrocarbons (about 42%), esters (about 39%), wax acids and wax alcohols. It can be obtained, for example, from the comminuted fleshy leaves of a thornless spurge species (Euphorbia cerifera) by boiling with aqueous sulfuric acid.

Carnauba wax is a yellowish, greenish or dark-gray mass which can be obtained in varying grades, obtained by selection, from the leaves of the Brazilian fan palm Copernicia prunifera or carnauba palm (Carnauba cerifera) by, for example, brushing the wax dust from the withered fronds, melting it and filtering it and, after solidification, breaking it into pieces. Canauba wax can be lightened by bleaching agents. It contains about 85% esters, in each case about 2-3% free wax acids (carnaubic, behenic, lignoceric, melissic and cerotic acid), long-chain alcohols, diols and saturated hydrocarbons.

Japan wax (also: Japan tallow or Cera japonica) is colorless or yellowish, pure plant fat which can be obtained, for example, in Japan from the fruits of a tree-like sumac plant (Rhus succedanea) by boiling. The main constituents of japan wax are palmitic glycerol esters and also esters of japanic acid (heneicosanedioic acid, C₂₁H₄₀O₄), of phellogenic acid (docosanedioic acid, C₂₂H₄₂O₄) and of tricosanedioic acid (C₂₃H₄₄O₄).

Esparto wax is obtained as a by-product in the manufacture of pulp and paper from the esparto grass (Graminaceae) indigenous to Mediterranean countries. It consists of about 15 to 17% of wax acids (e.g. cerotic and melissic acid), of 20 to 22% of alcohols and hydrocarbons, and of 63 to 65% of esters.

Particularly advantageous natural waxes for the purposes of the present invention are, for example, those available under the trade names Permulgin 1550 and Permulgin 4002 from KOSTER KEUNEN, and those available under the trade names Shellac Wax 7302 L and Candelilla Wax 2039 L from KAHL wax refinery.

Also advantageous according to the invention are chemically modified waxes and synthetic waxes. Preferred modified waxes are, for example, beeswax esters, in particular the alkyl beeswaxes available under the trade names BW Ester BW 67, BW Ester BW 80 from KOSTER KEUNEN.

Preferred synthetic waxes are, for example, that available under the trade name beeswax component B 85 from SCHLICKUM, and silicone-based waxes, such as, for example, dialkoxydimethylpolysiloxanes, which are characterized by the following structure

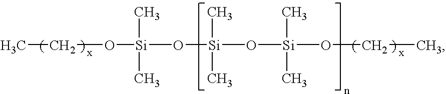

in which x is a number between 18 and 24. Behenoxydimethicone, for which x from the above structural formula is 21 and which is available under the trade name Abil® Wax 2440 from Th. Goldschmidt AG, is particularly advantageous. Also preferred according to the invention is a silicone-based wax available under the trade name Siliconyl Beeswax from KOSTER KEUNEN.

Further advantageous synthetic waxes are certain fatty acids and/or fatty acid mixtures, for example C₁₆₋₃₆-fatty acids, in particular those available under the trade name Syncrowax AW1C from Croda GmbH.

Also advantageous for the purposes of the present invention are ester waxes, which are esters of

1. a saturated and/or unsaturated, branched and/or unbranched mono- and/or dicarboxylic acid having 12 to 40 carbon atoms and

2. a saturated and/or unsaturated, branched and/or unbranched alcohol having 12 to 40 carbon atoms.

Particularly advantageous ester waxes are those chosen from the group listed below:

| Ester wax | Trade name | available from |
|---|---|---|
| Myristyl myristate | Cetiol MM | Henkel KGaA |
| Cetyl palmitate | Cutina CP | Henkel KGaA |
| Cetyl ricinoleate | Tegosoft CR | Goldschmidt |
| C₁₄₋₃₄ Alkyl stearate | Kester wax K 76 H | KOSTER KEUNEN |
| C₂₀₋₄₀ Dialkyl dimerate | Kester wax K 80 D | KOSTER KEUNEN |
| Ditetracosanyl dimerate | Kester wax K70D | KOSTER KEUNEN |
| C₁₆₋₃₈ Alkyl hydroxystearoyl | Kester wax K80P | KOSTER KEUNEN |
| stearate | | |
| C₂₀₋₄₀ Alkyl stearoylstearoyl | Kester wax | KOSTER KEUNEN |
| stearate | K80P-VS | |
| C₂₀₋₄₀ Alkylstearate | Kester wax K 82 | KOSTER KEUNEN |
| Hydroxystearyl | Elfacos C26 | AKZO NOBEL |
| hydroxystearate | | |

For the purposes of the present invention, particular preference is given to cetyl ricinoleate.

Also advantageous are esters of glycol, in particular glycol esters of lignoceric acid (CH₃(CH₂)₂₂COOH), of cerotic acid (CH₃(CH₂)₂₄COOH) and/or of montanic acid (CH₃(CH₂)₂₆COOH). Very particularly advantageous for the purposes of the present invention are glycol esters of montanic acid (CH₃(CH₂)₂₆COOH). An advantageous glycol montanate is available, for example, in a mixture with butylene glycol montanate under the trade name Wax E Pharma from Clariant.

It is also advantageous to choose the wax components from the group of glycerides, in particular from the group of triglycerides. The glycerides and triglycerides listed below are particularly advantageous:

| Glyceride | Trade name | available from |
|---|---|---|
| C₁₆₋₁₈ Triglyceride | Cremeol HF-52- | Aarhus Oliefabrik |
| | SPC | |
| Glyceryl hydroxystearate | Naturchem GMHS | Rahn |
| Hydrogenated cocoglycerides | Softisan 100 | Huls AG |
| Caprylic/capric/isostearic/adipic | Softisan 649 | Dynamit Nobel |
| triglyceride | | |
| C₁₈₋₃₆ Triglyceride | Syncrowax HGLC | Croda GmbH |
| Glyceryl tribehenate | Syncrowax HRC | Croda GmbH |
| Glyceryl tri(12-hydroxystearate) | Thixcin R | Rheox/NRC |
| Hydrogenated castor oil | Cutina HR | Henlek KGaA |
| C₁₆₋₂₄ Triglyceride | Cremeol HF-62- | Aarhus Oliefabrik |
| | SPC | |

Also of particular preference for the purposes of the present invention is shea butter, also called karite oil or galam butter (CAS No. 68920-03-6). Shea butter is the fat of the seeds or kernels of the plant Butyrospermum Parkii belonging to the family of the Sapotaceae, and consists of approximately 34 to 45% by weight of solid fatty acids (principally stearic acid) and of approximately 50 to 60% by weight of liquid fatty acids (principally comprising oleic acid).

According to the invention, the waxes are also preferably chosen from the group of saturated and/or unsaturated, branched and/or unbranched fatty alcohols having 14 to 40 carbon atoms, particular preference being given to behenyl alcohol (C₂₂H₄₅OH), cetearyl alcohol [a mixture of cetyl alcohol (C₁₆H₃₃OH) and stearyl alcohol (C₁₈H₃₇OH)], cetyl arachidol [2-hexadecyl-1-eicosanol (C₃₆H₇₃OH), and/or 2-tetradecyloctadecanol (C₃₂H₆₅OH). Advantageous embodiments of the last two mentioned fatty alcohols are available under the trade names Isofol 36 and Isofol 32 from Condea.

Further advantageous waxes for the purposes of the present invention are:

paraffin waxes or mineral waxes, such as ceresin, ozokerite and the like,

animal waxes, such as spermaceti, lanolin (wool wax), uropygial grease and the like,

chemically modified waxes, hard waxes, such as montan ester waxes, sasol waxes, hydrogenated jojoba waxes

synthetic waxes

polyalkylene waxes, polyethylene glycol waxes

Particularly advantageous for the purposes of the present invention is, for example, microwax, a wax product from the mineral wax which sediments in crude oil storage tanks and in oil pipelines, and is present in lubricating oil distillates and in distillation residues of heavy crude oils, which is deoiled and lightened by treatment with sulfuric acid, aluminum chloride or bleaching earth.

In contrast to coarsely crystalline commercial paraffin, microwax has a very fine crystalline structure; it is therefore sometimes also referred to a microcrystalline paraffin (microparaffin), ceresin or hard petrolatum and is similar to ozokerite in its properties. It is advantageous for the purposes of the present invention to use microwax in mixtures with conventional paraffin and/or further waxes.

Also advantageous for the purposes of the present invention are ozokerites (earth waxes), pale yellow, brown, gray-black or black, amorphous, ointment-like soft to brittle, hard masses with melting points between 50 and 100° C. The hard, high-melting types are virtually odorless, the softer ones in some cases having a pleasant aromatic odor, in some cases like crude oil. The composition of the ozokerites is not uniform; typical constituents are paraffins (e.g. evenkit=tetracosane) and isoparaffins (<C₃₅), lower and polycyclic aromatics, mono- and bicyclic naphthenes with or without side chains, and small amounts of alcohols, esters, porphyrins and trace elements.

Ceresin is also an advantageous wax for the purposes of the present invention. Ceresin (mineral wax, cera mineralis alba) is a colorless, odorless, tasteless wax with a melting point of from 58 to 80° C. (purified 70 to 75⁶C). Ceresin is a mixture of normal, branched-chain and cyclic saturated hydrocarbons which is obtained by refining ozokerite earth wax (hydrocarbon wax) of the fossil origin.

Further advantageous waxes for the purposes of the present invention are paraffin waxes. Paraffin waxes is the term used for solid mixtures of purified, saturated aliphatic hydrocarbons (paraffins) which are colorless, odorless and tasteless. Also advantageous is hard paraffin (paraffinum solidum), a solid crystalline mass. For semisolid paraffin wax grades with melting points from 45 to 65° C., names such as soft paraffin are in use, and for those with melting points from 38 to 60° C. and boiling points >300° C., names such as petrolatum are in use; a known trade name for the latter is Vaseline. Vaseline is an advantageous wax for the purposes of the present invention. Vaseline is available as yellow and white Vaseline and is a mineral fat.

It is advantageous for the purposes of the present invention to choose the total amount of the waxes and/or lipids from the range from 0.05 to 10% by weight, advantageously from 0.1 to 5% by weight, in each case based on the total weight of the formulation.

Particularly advantageous preparations for the purposes of the present invention comprise an emulsifier system which consists of

A. at least one emulsifier A chosen from the group of completely, partially or unneutralized, branched and/or unbranched, saturated and/or unsaturated fatty acids with a chain length of from 10 to 40 carbon atoms,

B. at least one emulsifier B chosen from the group of polyethoxylated fatty acid esters with a chain length of from 10 to 40 carbon atoms and with a degree of ethoxylation of from 5 to 100 and

C at least one coemulsifier C chosen from the group of saturated and/or unsaturated, branched and/or unbranched fatty alcohols with a chain length of from 10 to 40 carbon atoms.

The emulsifier(s) A is/are preferably chosen from the group of fatty acids which have been completely or partially neutralized with customary alkalis (such as, for example, sodium hydroxide and/or potassium hydroxide, sodium carbonate and/or potassium carbonate, and mono- and/or triethanolamine). Stearic acid and stearates, isostearic acid and isostearates, palmitic acid and palmitates, and myristic acid and myristates, for example, are particularly advantageous.

The emulsifier(s) B is/are preferably chosen from the following group: PEG-9 stearate, PEG-8 distearate, PEG-20 stearate, PEG-8 stearate, PEG-8 oleate, PEG-25 glyceryl trioleate, PEG-40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methylglucose sesquistearate, PEG-30 glyceryl isostearate, PEG-20 glyceryl laurate, PEG-30 stearate, PEG-30 glyceryl stearate, PEG-40 stearate, PEG-30 glyceryl laurate, PEG-50 stearate, PEG-100 stearate, PEG-150 laurate. Particularly advantageous are, for example, polylethoxylated stearic esters.

The coemulsifier(s) C is/are preferably chosen according to the invention from the following group: behenyl alcohol (C₂₂H₄₅OH), cetearyl alcohol [a mixture of cetyl alcohol (C₁₆H₃₃OH) and stearyl alcohol (C₁₈H₃₇OH)], lanolin alcohols (wool wax alcohols, which are the unsaponifiable alcohol fraction of wool wax which is obtained following the saponification of wool wax). Particular preference is given to cetyl alcohol and cetylstearyl alcohol.

It is advantageous according to the invention to choose the weight ratios of emulsifier A to emulsifier B to coemulsifier C (A:B:C) as a:b:c, where a, b and c, independently of one another, may be rational numbers from 1 to 5, preferably from 1 to 3. Particular preference is given to a weight ratio of approximately 1:1:1.

It is advantageous for the purposes of the present invention to choose the total amount of emulsifiers A and B and of coemulsifier C from the range from 2 to 20% by weight, advantageously from 5 to 15% by weight, in particular from 7 to 13% by weight, in each case based on the total weight of the formulation.

For the purposes of the present invention, it is particularly preferred if the gas phase of the preparations comprises carbon dioxide or consists entirely of carbon dioxide. It is particularly advantageous if carbon dioxide is a or the active ingredient in the preparations according to the invention.

Compositions according to the invention develop, even during their preparation—for example during stirring or upon homogenization—into fine-bubble foams. According to the invention, fine-bubble, rich foams of excellent cosmetic elegance are obtainable. Furthermore, preparations which are particularly well tolerated by the skin are obtainable according to the invention, where valuable ingredients can be distributed on the skin in a particularly good manner.

It may be advantageous, although it is not necessary, for the formulations according to the present invention to comprise further emulsifiers. Preference is given to using those emulsifiers which are suitable for the preparation of W/O emulsions, it being possible for these to be present either individually or else in any combinations with one another.

Preferably, for the purposes of the present invention, the further emulsifier(s) is/are chosen from the group of hydrophilic emulsifiers. According to the invention, particular preference is given to mono-, di- and tri-fatty acid esters of sorbitol.

The total amount of further emulsifiers is, according to the invention, advantageously chosen to be less than 5% by weight, based on the total weight of the formulation.

The list of given further emulsifiers which can be used for the purposes of the present invention is not of course intended to be limiting.

Particularly advantageous preparations for the purposes of the present invention are free from mono- or diglyceryl fatty acid esters. Particular preference is given to preparations according to the invention which comprise no glyceryl stearate, glyceryl isostearate, glyceryl diisostearate, glyceryl oleate, glyceryl palmitate, glyceryl myristate, glyceryl lanolate and/or glyceryl laurate.

The oil phase of the preparations according to the invention is advantageously chosen from the group of nonpolar lipids having a polarity ≧30 mN/m and of the cyclic or linear silicone oils. Particularly advantageous nonpolar lipids for the purposes of the present invention are those listed below.

| | | | Polarity |
|---|---|---|---|
| Manufacturer | Trade name | INCI name | mN/m |
| Total SA | Ecolane 130 | Cycloparaffin | 49.1 |
| Neste PAO N.V. | Nexbase 2006 FG | Polydecene | 46.7 |
| (Supplier Hansen & Rosenthal) | | | |
| Chemische Fabrik Lehrte | Polysynlane | Hydrogenated | 44.7 |
| | | Polyisobutene | |
| Wacker | Wacker Silicone | Polydimethylsiloxane | 46.5 |
| | oil AK 50 | | |
| EC Erdolchemie (Supplier | Solvent ICH | Isohexadecane | 43.8 |
| Bayer AG) | | | |
| DEA Mineral oil (Supplier | Pionier 2076 | Mineral Oil | 43.7 |
| Hansen & Rosenthal) | | | |
| Tudapetrol | | | |
| DEA Mineral oil (Supplier | Pionier 6301 | Mineral Oil | 43.7 |
| Hansen & Rosenthal) | | | |
| Tudapetrol | | | |
| Wacker | Wacker Silicone | Polydimethylsiloxane | 42.4 |
| | oil AK 35 | | |
| EC Erdolchemie GmbH | Isoeicosane | Isoeicosane | 41.9 |
| Wacker | Wacker Silicone | Polydimethylsiloxane | 40.9 |
| | oil AK 20 | | |
| Condea Chemie | Isofol 1212 | | 40.3 |
| | Carbonate | | |
| Gattefosse | Softcutol O | Ethoxydiglycol Oleate | 40.5 |
| Creaderm | Lipodermanol OL | Decyl Olivate | 40.3 |
| Henkel | Cetiol S | Dioctylcyclohexane | 39.0 |
| DEA Mineral oil (Supplier | Pionier 2071 | Mineral Oil | 38.3 |
| Hansen & Rosenthal) | | | |
| Tudapetrol | | | |
| WITCO BV | Hydrobrite 1000 | Paraffinum Liquidum | 37.6 |
| | PO | | |
| Goldschmidt | Tegosoft HP | Isocetyl Palmitate | 36.2 |
| Condea Chemie | Isofol Ester 1693 | | 33.5 |
| Condea Chemie | Isofol Ester 1260 | | 33.0 |
| Dow Corning | Dow Corning Fluid | Cyclopentasiloxane | 32.3 |
| | 245 | | |
| Unichema | Prisorine 2036 | Octyl Isostearate | 31.6 |
| Henkel Cognis | Cetiol CC | Dicaprylyl Carbonate | 31.7 |
| ALZO (ROVI) | Dermol 99 | Trimethylhexyl | 31.1 |
| | | Isononanoate | |
| ALZO (ROVI) | Dermol 89 | 2-Ethylhexyl | 31.0 |
| | | Isononanoate | |
| Unichema | Estol 1540 EHC | Octyl Cocoate | 30.0 |

Of the hydrocarbons, paraffin oil, and further hydrogenated polyolefins, such as hydrogenated polyisobutenes, squalane and squalene, in particular, are to be used advantageously for the purposes of the present invention.

The content of the lipid phase is advantageously chosen to be less than 50% by weight, preferably between 2.5 and 30% by weight, particularly preferably between 5 and 15% by weight, in each case based on the total weight of the preparation. It may also be advantageous, although it is not obligatory, for the lipid phase to comprise up to 40% by weight, based on the total weight of the lipid phase, of polar lipids (having a polarity of <20 mN/m) and/or medium-polarity lipids (having a polarity of from 20 to 30 mN/m).

For the purposes of the present invention, particularly advantageous polar lipids are all native lipids, such as, for example, olive oil, sunflower oil, soybean oil, groundnut oil, rapeseed oil, almond oil, palm oil, coconut oil, castor oil, wheatgerm oil, grapeseed oil, thistle oil, evening primrose oil, macadamia nut oil, corn oil, avocado oil and the like and those listed below.

| | | | Polarity |
|---|---|---|---|
| Manufacturer | Trade name | INCI name | mN/m |
| Condea Chemie | Isofol 14 T | Butyl Decanol (+) Hexyl | 19.8 |
| | | Octanol (+) | |
| | | Hexyl Decanol (+) Butyl | |
| | | Octanol | |
| Lipochemicals | Lipovol MOS- | Tridecyl Stearate(+) | 19.4 |
| INC./USA | 130 | Tridecyl Trimellitate(+) | |
| (Induchem) | | Dipentaerythrityl | |
| | | Hexacaprylate/Hexacaprate | |
| | Castor oil | | 19.2 |
| CONDEA | Isofol Ester | | 19.1 |
| Chemie | 0604 | | |
| Huels | Miglyol 840 | Propylene Glycol | 18.7 |
| CONDEA | | Dicaprylate/Dicaprate | |
| Chemie | | | |
| CONDEA | Isofol 12 | Butyl Octanol | 17.4 |
| Chemie | | | |
| Goldschmidt | Tegosoft SH | Stearyl Heptanoate | 17.8 |
| | Avocado oil | | 14.5 |
| Henkel Cognis | Cetiol B | Dibutyl Adipate | 14.3 |
| ALZO (ROVI) | Dermol 488 | PEG 2 Diethylene | 10.1 |
| | | Hexanoate | |
| Condea Augusta | Cosmacol ELI | C₁₂₋₁₃ Alkyl Lactate | 8.8 |
| S.P.A. | | | |
| ALZO (ROVI) | Dermol 489 | Diethylene Glycol | 8.6 |
| | | Dioctanoate(/ | |
| | | Diisononanoate | |
| Condea Augusta | Cosmacol ETI | Di-C_{12/13} Alkyl Tartrate | 7.1 |
| S.P.A. | | | |
| Henkel Cognis | Emerest 2384 | Propylene Glycol | 6.2 |
| | | Monoisostearate | |
| Henkel Cognis | Myritol 331 | Cocoglycerides | 5.1 |
| Unichema | Prisorine 2041 | Triisostearin | 2.4 |
| | GTIS | | |

Particularly advantageous medium-polar lipids for the purposes of the present invention are those listed below

| | | | Polarity |
|---|---|---|---|
| | | | (Water) |
| Manufacturer | Trade name | INCI name | mN/m |
| Henkel Cognis | Cetiol OE | Dicaprylyl Ether | 30.9 |
| | Dihexyl | Dihexyl Carbonate | 30.9 |
| | carbonate | | |
| Albemarle S.A. | Silkflo 366 NF | Polydecene | 30.1 |
| Stearinerie | DUB VCI 10 | Isodecyl Neopentanoate | 29.9 |
| Dubois Fils | | | |
| ALZO (ROVI) | Dermol IHD | Isohexyl Decanoate | 29.7 |
| ALZO (ROVI) | Dermol 108 | Isodecyl Octanoate | 29.6 |
| | Dihexyl Ether | Dihexyl Ether | 29.2 |
| ALZO (ROVI) | Dermol 109 | Isodecyl 3,5,5 Trimethyl | 29.1 |
| | | Hexanoate | |
| Henkel Cognis | Cetiol SN | Cetearyl Isononanoate | 28.6 |
| Unichema | Isopropyl | Isopropyl Palmitate | 28.8 |
| | palmitate | | |
| Dow Corning | DC Fluid 345 | Cyclomethicone | 28.5 |
| Dow Corning | Dow Corning | Cyclopolydimethylsiloxane | 28.5 |
| | Fluid 244 | | |
| Nikko | Jojoba oil Gold | | 26.2 |
| Chemicals | | | |
| Superior Jojoba | | | |
| Oil Gold | | | |
| Wacker | Wacker AK 100 | Dimethicone | 26.9 |
| ALZO (ROVI) | Dermol 98 | 2-Ethylhexanoic Acid 3,5,5 | 26.2 |
| | | Trimethyl Ester | |
| Dow Corning | Dow Corning | Open | 25.3 |
| | Fluid 246 | | |
| Henkel Cognis | Eutanol G | Octyldodecanol | 24.8 |
| Condea | Isofol 16 | Hexyl Decanol | 24.3 |
| Chemie | | | |
| ALZO (ROVI) | Dermol 139 | Isotridecyl 3,5,5 | 24.5 |
| | | Trimethylhexanonanoate | |
| Henkel Cognis | Cetiol PGL | Hexyldecanol (+) Hexyl | 24.3 |
| | | Decyl Laurate | |
| | Cegesoft C24 | Octyl Palmitate | 23.1 |
| Gattefosse | M.O.D. | Octyldodeceyl Myristate | 22.1 |
| | Macadamia Nut | | 22.1 |
| | Oil | | |
| Bayer AG, | Silicone oil VP | Phenyl Trimethicone | 22.7 |
| Dow Corning | 1120 | | |
| CONDEA | Isocarb 12 | Butyl Octanoic Acid | 22.1 |
| Chemie | | | |
| Henkel Cognis | Isopropyl | Isopropyl Stearate | 21.9 |
| | stearate | | |
| WITCO, | Finsolv TN | C12-15 Alkyl Benzoate | 21.8 |
| Goldschmidt | | | |
| Dr. Straetmans | Dermofeel BGC | Butylene Glycol | 21.5 |
| | | Caprylate/Caprate | |
| Unichema | Miglyol 812 | Caprylic/Capric | 21.3 |
| Huels | | Triglyceride | |
| Trivent (via S. | Trivent OCG | Tricaprylin | 20.2 |
| Black) | | | |
| ALZO (ROVI) | Dermol 866 | PEG,, Diethylhexanoate/ | 20.1 |
| | | Diisononanoate/Ethylhexyl | |
| | | Isononanoate | |

The cosmetic or dermatological preparations according to the invention can also advantageously comprise inorganic particulate hydrophobic, hydrophobicized or oil-absorbing solid-body substances or inorganic gel formers.

Advantageous inorganic particulate hydrophobic, hydrophobicized or oil-absorbing solid-body substances may, for example, be chosen from the group of

inorganic fillers (such as talc, kaolin, zeolites, boron nitride),

inorganic pigments based on metal oxides and/or other metal compounds which are sparingly soluble or insoluble in water (in particular oxides of titanium, zinc, iron, manganese, aluminum, cerium),

inorganic pigments based on silicon oxides (such as, in particular, the grades Aerosil-200, Aerosil 200 V), and

silicate derivatives (such as sodium silicoaluminates or fluoro magnesium silicates (submica grades), calcium aluminum borosilicates). Preference is given here in particular to silica dimethyl silylate (Aerosil® R972).

Aerosils [(fumed silica)=silicon dioxide obtained by thermal decomposition of ethyl silicate)] are highly disperse silicas with an often irregular shape, whose specific surface area is usually very large (200-400 m2/g) and can be controlled depending on the preparation process.

Aerosils to be used particularly advantageously according to the invention are obtainable, for example, under the trade names: Aerosil® 130 (Degussa Hüls) Aerosil® 200 (Degussa Hüls) Aerosil® 225 (Degussa Hüls) Aerosil® 300 (Degussa Hüls) Aerosil® 380 (Degussa Hüls) B-6C (Suzuki Yushi) CAB-O-SIL Fumed Silica (Cabot) CAB-O-SIL EH-5 (Cabot) CAB-O-SIL HS-5 (Cabot) CAB-O-SIL LM-130 (Cabot) CAB-O-SIL MS-55 (Cabot) CAB-O-SIL M-5 (Cabot) E-6C (Suzuki Yushi) Fossil Flour MBK (MBK) MSS-500 (Kobo) Neosil CT 11 (Crosfield Co.) Ronasphere (Rona/EM Industries) Silica, Anhydrous 31 (Whittaker, Clark & Daniels) Silica, Crystalline 216 (Whittaker, Clark & Daniels) Silotrat-1 (Vevy) Sorbosil AC33 (Crosfield Co.) Sorbosil AC 35 (Crosfield Co.) Sorbosil AC 37 (Crosfield Co.) Sorbosil AC 39 (Crosfield Co.) Sorbosil AC77 (Crosfield Co.) Sorbosil TC 15 (Crosfield Co.) Spherica (Ikeda) Spheriglass (Potters-Ballotini) Spheron L-1500 (Presperse) Spheron N-2000 (Presperse) Spheron P-1500 (Presperse) Wacker HDK H 30 (Wacker-Chemie) Wacker HDK N 20 (Wacker-Chemie) Wacker HDK P 100H (Wacker Silicones) Wacker HDK N 20P (Wacker-Chemie) Wacker HDK N 25P (Wacker-Chemie) Wacker HDK S 13 (Wacker-Chemie) Wacker HDK T 30 (Wacker-Chemie) Wacker HDK V 15 (Wacker-Chemie) Wacker HDK V 15 P (Wacker Chemie) Zelec Sil (DuPont).

It is also advantageous to use those SiO₂ pigments in which the free OH groups on the surface of the particles have been (completely or partially) organically modified. This gives, for example as a result of the addition of dimethylsilyl groups, silica dimethyl silylate (e.g. Aerosil® R972 (Degussa Hüls) Aerosil® R974 (Degussa Hüls) CAB-O-SIL TS-610 (Cabot) CAB-O-SIL TS-720 (Cabot) Wacker HDK H15 (Wacker-Chemie) Wacker HDK H18 (Wacker-Chemie) Wacker HDK H₂O (Wacker-Chemie)). The addition of trimethylsilyl groups gives silica silylate (e.g. Aerosil R 812 (Degussa HUls) CAB-O-SIL TS-530 (Cabot) Sipernat D 17 (Degussa Hüls) Wacker HDK H2000 (Wacker-Chemie)).

Very advantageous inorganic gel formers can, for example, be chosen from the group of modified or unmodified, naturally occurring or synthetic sheet silicates. Although it is entirely favorable to use pure components, the preparations according to the invention may also advantageously comprise mixtures of different modified and/or unmodified sheet silicates.

Sheet silicates, which are so-called phyllosilicates, are understood for the purposes of this application as meaning silicates and alumosilicates in which the silicate or aluminate units, respectively, are joined together via three Si—O or Al—O bonds and form a waved sheet or layer structure. The fourth Si—O or Al—O valence is saturated by cations. There are relatively weak electrostatic interactions, e.g. hydrogen bridge bonds, between the individual layers. The layer structure is consequently defined largely by strong covalent bonds.

The stoichiometery of the sheet silicates is (Si₂O₃²⁻) for pure silicate structures and (AlₘSi²⁻ₘO₅(^{2+m})⁻) for alumosilicates, wherein m is a number greater than zero and less than 2.

If no pure silicates are present, but alumosilicates, it should be taken into consideration that each Si⁴⁺ group replaced by Al³⁺ requires a further singly charged cation to neutralize the charge.

The charge balance is preferably balanced by H⁺, alkali metal or alkaline earth metal ions. Aluminum as counterion is also known and advantageous. In contrast to the alumosilicates, these compounds are called aluminum silicates. “Aluminum alumosilicates”, in which aluminum is present both in the silicate network, and also as counterion, are also known and in some cases advantageous for the present invention.

Sheet silicates are well documented in the literature, e.g. in the “Lehrbuch der Anorganischen Chemie” [Textbook of inorganic chemistry], A. F. Hollemann, E. Wiberg and N. Wiberg, 91st-100th edition, Walter de Gruyter—Verlag 1985, passim, and also “Lehrbuch der Anorganischen Chemie” [Textbook of inorganic chemistry], H. Remy, 12th edition, Akademische Verlagsgesellschaft, Leipzig 1965, passim. The layer structure of montmorillonite can be found in Rompps Chemie-Lexikon, Franckh'sche Verlagshandlung W. Keller & Co., Stuttgart, 8th edition, 1985, p. 2668 f.

Examples of sheet silicates are:

| | |
|---|---|
| Montmorilonite | Na_{0.33}((Al_{1.67}Mg_{0.33})(OH)₂(Sᵢ₄O₁₀)), often simplified to |
| | Al₂O₃*4SiO₂*H₂O*nH₂O or Al₂[(OH)₂/Si₄O₁₀].n H₂O |
| Kaolinite | Al₂(OH)₄(Si₂O₅) |
| Ilite | (K₁H₃O)_{y}(Mg₃(OH)₂(Si_{4-y}Al_{y}O₁₀)) and |
| | (K₁H₃O)_{y}(Al₂(OH)₂(Si_{4-y}Al_{y}O₁₀)) |
| | where y = 0.7-0.9 |
| Beidelite | (Ca, Na)_{0.3}(Al₂(OH)₂(Al_{0.5}Si_{3.5}O₁₀)). |
| Nontronite | Na_{0.33}(Fe₂(OH)₂(Al_{0.33}S_{i3.67}O₁₀)) |
| Saponite | (Ca, Na)_{0.33}(Mg, Fe)₃(OH)₂(Al_{0.33}Si_{3.67}O₁₀)) |
| Hectorite | Na_{0.33}(Mg, Li)₃(OH, F)₂(Si₄O₁₀)) |

Montmorillonite represents the main mineral of the naturally occurring bentonites.

Very advantageous inorganic gel formers for the purposes of the present invention are aluminum silicates, such as the montmorillonites (bentonites, hectorites and derivatives thereof, such as quaternium-18 bentonite, quaternium-18 hectorite, stearalkonium bentonite and stearalkonium hectorite) or however magnesium aluminum silicates (Veegum® grades), and sodium magnesium silicates (Laponite® grades).

Montmorillonites represent clay minerals which belong to the dioctahedral smectites, and are masses which swell in water, but do not become plastic. The layer packets in the three-layer structure of the montmorillonites can swell as the result of reversible incorporation of water (in a 2- to 7-fold amount) and other substances such as, for example, alcohols, glycols, pyridine, α-picoline, ammonium compounds, hydroxy-aluminosilicate ions etc.

The chemical formula given above is only approximate; since montmorillonite has a large capacity for ion exchange, Al can be replaced by Mg, Fe²⁺, Fe³⁺, Zn, Pb, Cr, and also Cu and others. The resulting negative charge of the octahedral layers is balanced by cations, in particular Na⁺ (sodium montmorillonite) and Ca²⁺ (calcium montmorillonite is only swellable to a very small degree) in interlayer positions.

Synthetic magnesium silicates and/or bentonites advantageous for the purposes of the present invention are sold, for example, by Sud-Chemie under the tradename Optigel®.

An aluminum silicate advantageous for the purposes of the present invention is sold, for example, by R. T. Vanderbilt Comp., Inc., under the trade name Veegum®. The various Veegum® grades, which are all advantageous according to the invention, are characterized by the following compositions

| | (regular grade) | HV | K | HS | S-728 |
|---|---|---|---|---|---|
| SiO₂ | 55.5 | 56.9 | 64.7 | 69.0 | 65.3 |
| MgO | 13.0 | 13.0 | 5.4 | 2.9 | 3.3 |
| Al₂O₃ | 8.9 | 10.3 | 14.8 | 14.7 | 17.0 |
| Fe₂O₃ | 1.0 | 0.8 | 1.5 | 1.8 | 0.7 |
| CaO | 2.0 | 2.0 | 1.1 | 1.3 | 1.3 |
| Na₂O | 2.1 | 2.8 | 2.2 | 2.2 | 3.8 |
| K₂O | 1.3 | 1.3 | 1.9 | 0.4 | 0.2 |
| Ashing loss | 11.1 | 12.6 | 7.6 | 5.5 | 7.5 |

These products swell in water to form viscous gels, which have an alkaline reaction. The organophilization of montmorillonite or bentonites (exchange of the interlayer cations for quaternary alkylammonium ions) produces products (bentones) which are preferably used for dispersion in organic solvents and oils, fats, ointments, paints, coatings and in detergents.

Bentone® is a trade name for various neutral and chemically inert gelling agents which are constructed from long-chain, organic ammonium salts and specific types of montmorillonite.

The following Bentone® grades are sold, for example, from Kronos Titan and are to be used advantageously for the purposes of the present invention: Bentone® 27, an organically modified montmorillonite, Bentone® 34 (dimethyldioctylammonium bentonite), which is prepared in accordance with U.S. Pat. No. 2,531,427 and, because of its lipophilic groups, swells more readily in a lipophilic medium than in water, Bentone® 38, an organically modified montmorillonite, a cream-colored to white powder, Bentone® LT, a purified clay mineral, Bentone® Gel MIO, an organically modified montmorillonite which is supplied as a very fine suspension in mineral oil (SUS-71) (10% bentonite, 86.5% mineral oil and 3.3% wetting agent), Bentone® Gel IPM, an organically modified bentonite which is suspended in isopropyl myristate (10% bentonite, 86.7% isopropyl myristate, 3.3% wetting agent), Bentone® Gel CAO, an organically modified montmorillonite which is taken up in castor oil (10% bentonite, 86.7% castor oil and 3.3% wetting agent), Bentone® Gel Lantrol, an organically modified montmorillonite which, in paste form, is intended for the further processing, in particular for the preparation of cosmetic compositions; 10% bentonite, 64.9 Lantrol (wool wax oil), 22.0 isopropyl myristate, 3.0 wetting agent and 0.1 propyl p-hydroxybenzoate, Bentone® Gel Lan I, a 10% strength Bentone® 27 paste in a mixture of wool wax USP and isopropyl palmitate, Bentone® Gel Lan II, a bentonite paste in pure, liquid wool wax, Bentone® Gel NV, a 15% strength Bentone® 27 paste in dibutyl phthalate, Bentone® Gel OMS, a bentonite paste in Shellsol T., Bentone® Gel OMS 25, a bentonite paste in isoparaffinic hydrocarbons (Idopar® H), Bentone® Gel IPP, a bentonite paste in isopropyl palmitate.

All Bentone grades are to be used advantageously for the purposes of the present invention.

For the purposes of the present invention, preparations may also advantageously comprise one or more hydrocolloids from one or more of the following groups:

organic, natural compounds, such as, for example, agar agar, carrageen, tragacanth, gum arabic, alginates, pectins, polyoses, guar flour, carob bean flour, starch, dextrins, gelatin, casein,

organic, modified natural substances, such as, for example, carboxymethylcellulose and other cellulose ethers, hydroxyethyl cellulose and hydroxypropyl cellulose and microcrystalline celluose the like,

organic, completely synthetic compounds, such as, for example, polyacrylic and polymethacrylic compounds, vinyl polymers, polycarboxylic acids, polyethers, polyimines, polyamides, polylurethanes.

In addition, the preparations according to the present invention may advantageously comprise copolymers which are characterized by the following structural formula (I)

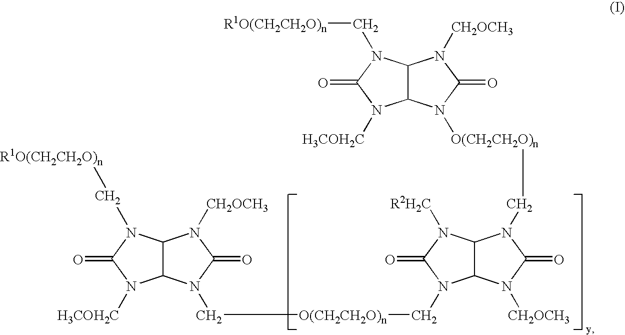

in which

R¹ is a branched or unbranched, saturated or unsaturated alkyl radical having 4 to 40 carbon atoms,

R²=—OCH₃ or —O(CH₂CH₂O)ₓR¹,

x is an integer from 1 to 100,

n is an integer from 100 to 250 and

y is on average 2 or 3.

Particularly advantageous copolymers for the purposes of the present invention are those for which n is an integer from 150 to 200. It is particularly advantageous for the purposes of the present invention if, moreover, R¹ is a branched or unbranched, saturated or unsaturated alkyl radical having 8 to 12 carbon atoms.

It is also advantageous for the purposes of the present invention if the average molar mass of the copolymers is between 30 000 and 50 000.

According to the invention, PEG-180/octoxynol-40/tetramethoxymethylglycoluril copolymers where R²=—O(CH₂CH₂O)₄₀C₈H₁₇ and n=180 are very particularly advantageous.

According to the invention, PEG-180/laureth-50/tetramethoxymethylglycoluril copolymers where R²=—O(CH₂CH₂O)₅₀C₁₂H₂₅ and n=180 are also particularly advantageous.

According to the invention, polyether-1 is also particularly advantageous.

The cosmetic and/or dermatological preparations according to the invention can have the customary composition. Particularly advantageous for the purposes of the present invention are skincare preparations: they can be used for cosmetic and/or dermatological light protection, and also for the treatment of the skin and/or of the hair and as a make-up product in decorative cosmetics. A further advantageous embodiment of the present invention consists in after-sun products.

Depending on their formulation, cosmetic or topical dermatological compositions for the purposes of the present invention can, for example, be used as skin protection cream, day or night cream etc. It is optionally possible and advantageous to use the compositions according to the invention as bases for pharmaceutical formulations.

Just as emulsions of liquid and solid consistency are used as cosmetic cleansing lotions or cleansing creams, the preparations according to the invention can also represent “cleansing foams”, which can be used, for example, for the removal of make-up or as a mild washing foam—optionally also for blemished skin. Such cleansing foams can also advantageously be applied as so-called “rinse off” preparations, which are rinsed off from the skin following application.

The cosmetic and/or dermatological preparations according to the invention can also advantageously be in the form of a foam for the care of hair and/or the scalp, in particular a foam for arranging the hair, a foam which is used while blow-drying the hair, a styling and treatment foam.

For use, the cosmetic and dermatological preparations according to the invention are applied to the skin and/or the hair in an adequate amount in the manner customary for cosmetics.

The cosmetic or dermatological compositions according to the invention can, for example, be removed from aerosol containers and thereby foamed. Aerosol containers according to the invention are spray devices with a filling of the liquid or slurry-like substances, which are under the pressure of a propellant (pressurized gas or aerosol packagings). Such containers can be provided with valves of very different construction which permit the removal of the contents in the form of a foam.

In addition, the preparations according to the present invention may advantageously be removed from propellent gas-free, mechanically operated pump atomizers (pump dispensers). Particularly advantageous for the purposes of the present invention are pump systems which work without pressurized gas, but with a filter which brings about special swirling.

The cosmetic or dermatological preparations according to the invention can also preferably be removed, for example, from two-chamber aerosol containers and be applied to the skin. Packaging means advantageous according to the invention are containers in which there is one chamber with a filling of the liquid or slurry-like preparations under the pressure of a primary propellant located in a second chamber. Such containers can be equipped with valves of very different construction which permit the removal of the contents of the first chamber in the form of an emulsion or gel in any position—even with the valve pointing downward. An advantageous embodiment are BiCan® aerosol containers in which the product is enclosed in a flexible bag made of metal or plastic within the can.

After-foaming compositions according to the invention represent unfoamed, i.e. directly after exiting an aerosol container, two- or multiphase systems—generally emulsions. They can be made into foams by gentle rubbing, for example in the hands or during application and rubbing on the skin, but also by stirring or other foaming operations.

Moreover, it has surprisingly been found that in the case of the use of (secondary) propellants, particularly advantageously of propellants soluble in the optionally present oil phase, thus, for example, customary propane/butane mixtures, the preparations according to the invention are not simply sprayed as aerosol droplets, but develop into finely-bubbled, rich foams as soon as such systems containing such (secondary) propellants experience decompression.

In the case of the use of hydrocarbons or mixtures thereof having 4 or 5 carbon atoms, in particular isobutane, n-pentane and isopentane, as (secondary) propellant, it is possible to delay the automatic foaming after exiting from the pressurized packaging.

As a result of the evaporation of the secondary propellant in the applied cosmetic product, heat is additionally withdrawn from the skin and a pleasant cooling effect is achieved. Such after-foaming preparations are therefore likewise regarded as advantageous embodiments of the present invention with independent inventive step.

Suitable pressurized gas containers for the purposes of the present invention are primarily cylindrical vessels made of metal (aluminum, tinplate, contents <1 000 ml), protected or shatter resistant glass or plastic (contents <220 ml) or shattering glass or plastic (contents <150 ml), in the choice of which compressive strength and breaking strength, corrosion resistance, ease of filling, or ease of sterilizing etc., but also esthetic aspects, handlability, printing properties etc. play a role. The maximum permissible operating pressure of spray cans made of metal at 50° C. is 12 bar and the maximum fill volume at this temperature is about 90% of the total volume. For glass and plastic cans, the values for the operating pressure are lower and dependent on the size of the container and the propellant (whether liquefied, compressed or dissolved gas).

For the purposes of the present invention, cans made of tinplate, aluminum and glass are particularly advantageous. For reasons of corrosion protection, metal cans can be coated on the inside (silver- or gold-coated), for which purpose all standard commercial internal protective coatings are suitable. For the purposes of the present invention, preference is given to polyester, epoxyphenol and polyamide-imide coatings. Film laminates made of polyethylene (PE), polypropylene (PP) and/or polyethylene terephthalate (PET) on the inside of the cans are also advantageous, in particular for cans made of tinplate.

The pressurized gas containers are usually single-part or two-part, but in most cases three-part cylindrical, conical or differently shaped. If plastics are used as the spray container material, then these should be resistant to chemicals and the sterilization temperature, gas-tight, impact-resistant and stable to internal pressures in excess of 12 bar. In principle, polyacetals and polyamides are suitable for spray container purposes.

The internal construction of the spray cans and the valve construction are many and varied, depending on the intended use and the physical nature of the ingredient—e.g. whether it is in the form of a two-phase or three-phase system—and can be determined by the person skilled in the art by simple trial and error without inventive activity. For suitable variants, reference may be made to the “Aerosol Technologie Handbuch der Aerosol-Verpackung” [Aerosol Technology Handbook of Aerosol Packaging] (Wolfgang Tauscher, Melcher Verlag GmbH Heidelberg/Munich, 1996).

Valves which are advantageous according to the invention can be designed with or without riser tube. The individual components from which valves according to the invention are usually constructed preferably consist of the following materials:

| | |
|---|---|
| Disk: | tinplate: uncoated, gold-or clear-coated, film-laminated (PE, PP or PET) |
| | aluminum: uncoated, silver-or gold-coated, different coating variants, Stoner- |
| | Mudge design |
| Seal: | natural or synthetic elastomers or thermoplastic (sleeve gaskets, film-lined |
| | made of PE or PP) internal and external seals, e.g. made of perbunan, buna, |
| | neoprene, butyl, CLB, LDPE, viton, EPDM, chlorobutyl, bromobutyl and/or |
| | diverse compounds |
| Cone: | PA, POM, brass and diverse special materials, |
| | standard bores (e.g.: 0.25 to 0.70 mm or 2 × 0.45 to 2 × 1.00 mm), |
| | various shaft diameters |
| Spring: | metal, particularly preferably V2A, stainless steel; |
| | plastic and also elastomer |
| Casing: | standard and impact |
| | VPH bores, RPT bores or slit for overhead applications |
| | materials: e.g. polyacetal, PA, PE, POM and the like |
| Riser tube: | plastic (polymer resin), e.g. PE, PP, PA or polycarbonate |

Advantageous spray heads for the purposes of the present invention are, for example, foaming heads for upright use (hold can vertically) or foam heads for overhead application using one or more channels.

Suitable propellants are the customary “classic” readily volatile, liquefied propellant gases, such as, for example, dimethyl ether (DME) and/or linear or branched-chain hydrocarbons with two to five carbon atoms (such as, in particular, ethane, propane, butane, isobutane and/or pentane), which can be used on their own or in a mixture with one another.

Compressed air, and also other gases which are under pressure, such as air, oxygen, nitrogen, hydrogen, helium, krypton, xenon, radon, argon, nitrous oxide (N₂O) and carbon dioxide (CO₂) are also advantageously to be used for the purposes of the present invention as propellant gases (either on their own or in any desired mixtures with one another).

The person skilled in the art is naturally aware that there are other propellant gases which are nontoxic per se and which would be suitable in principle for realizing the present invention in the form of aerosol preparations, but which nevertheless should be omitted due to an unacceptable impact on the environment or other accompanying circumstances, in particular halogenated (substituted by fluorine, chlorine, bromine, iodine and/or astatine) hydrocarbons, such as, for example, fluorocarbons and chlorofluorocarbons (CFCs).

For the purposes of the present invention, said gases can in each case be used individually or in any desired mixtures with one another.

For the purposes of the present invention, the volume fraction of propellant gas is advantageously chosen from the range from 0.1 to 30% by volume, based on the total volume of filler material and propellant gas (corresponding to a volume fraction of from 70 to 99.9% by volume of filler material).

A particularly preferred propellant gas for the purposes of the present invention is carbon dioxide. Foams obtainable from preparations according to the invention which comprise carbon dioxide as one or the active ingredient are particularly advantageous.

Particularly advantageous, finely creamy and rich foams are obtainable when the preparations according to the invention are foamed using linear or branched-chain, halogenated or nonhalogenated hydrocarbons. Very particularly advantageous foams are obtainable by foaming the preparations according to the invention with carbon dioxide, oxygen, compressed air, helium, krypton, xenon, radon, argon and/or nitrogen (either on their own or in any desired mixtures with one another).

The cosmetic and dermatological preparations according to the invention can comprise cosmetic auxiliaries, as are customarily used in such preparations, e.g. preservatives, preservative assistants, bactericides, perfumes, dyes, pigments which have a coloring effect, moisturizing and/or humectant substances, fillers which improve the feel on the skin, fats, oils, waxes or other customary constituents of a cosmetic or dermatological formulation, such as alcohols, polyols, polymers, foam stabilizers, electrolytes, organic solvents or silicone derivatives.

Advantageous preservatives for the purposes of the present invention are, for example, formaldehyde donors (such as, for example, DMDM hydantoin), iodopropyl butylcarbamates (e.g. those available under the trade names Koncyl-L, Koncyl-s and Konkaben LMB from Lonza), parabens, phenoxyethanol, ethanol, benzoic acid and the like. According to the invention, the preservative system usually also advantageously comprises preservative assistants, such as, for example, octoxyglycerol, glycine soya etc.

Particularly advantageous preparations are also obtained if antioxidants are used as additives or active ingredients. According to the invention, the preparations advantageously comprise one or more antioxidants. Favorable, but nevertheless optional antioxidants which may be used are all antioxidants customary or suitable for cosmetic or dermatological applications.

For the purposes of the present invention, water-soluble antioxidants, such as, for example, vitamins, e.g. ascorbic acid and derivatives thereof, can be used particularly advantageously.

Preferred antioxidants are also vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant(s), it is advantageous to choose their respective concentrations from the range from 0.001 to 10% by weight, based on the total weight of the formulation.

If vitamin A or vitamin A derivatives, or carotenes or derivatives thereof are the antioxidant(s), it is advantageous to choose their respective concentrations from the range from 0.001 to 10% by weight, based on the total weight of the formulation.

It is particularly advantageous when the cosmetic preparations according to the present invention comprise cosmetic or dermatological active ingredients, preferred active ingredients being antioxidants which can protect the skin against oxidative stress.

Further advantageous active ingredients for the purposes of the present invention are natural active ingredients and/or derivatives thereof, such as, for example, alpha-lipoic acid, phytoene, D-biotin, coenzyme Q10, alpha-glycosylrutin, carnitine, carnosine, natural and/or synthetic isoflavonoids, creatine, taurine and/or β-alanine.

Formulations according to the invention which comprise, for example, known antiwrinkle active ingredients, such as flavone glycosides (in particular α-glycosylrutin), coenzyme Q10, vitamin E and/or derivatives and the like are particularly advantageously suitable for the prophylaxis and treatment of cosmetic or dermatological changes in the skin, as arise, for example, during skin aging (such as, for example, dryness, roughness and the formation of dryness wrinkles, irritation, reduced refatting (e.g. after washing), visible vascular dilations (telangiectases, couperosis), sagging and formation of lines and wrinkles, local hyperpigmentation, hypopigmentation and abnormal pigmentation (e.g. age spots), increased susceptibility to mechanical stress (e.g. cracking) and the like. They are also advantageously suitable against the appearance of dry or rough skin.

Surprisingly, selected formulations according to the invention can also have an antiwrinkle effect or considerably increase the effect of known antiwrinkle active ingredients. Accordingly, formulations for the purposes of the present invention are particularly advantageously suitable for the prophylaxis and treatment of cosmetic or dermatological skin changes, as arise, for example, during skin aging. They are also advantageously suitable for combating the appearance of dry or rough skin.

In one particular embodiment, the present invention thus relates to products for the care of skin aged in a natural manner, and for the treatment of the secondary damage of photoaging, in particular the phenomena listed above.

The water phase of the preparations according to the invention can advantageously comprise customary auxiliaries, such as, for example, alcohols, in particular those of low carbon number, preferably ethanol and/or isopropanol, diols or polyols of low carbon number, and ethers thereof, preferably polyethylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes and moisturizers.

Moisturizers is the term used to describe substances or mixtures of substances which, following application or distribution on the surface of the skin, confer on cosmetic or dermatological preparations the property of reducing the moisture loss by the horny layer (also called transepidermal water loss (TEWL) and/or have a beneficial effect on the hydration of the horny layer.

Advantageous moisturizers for the purposes of the present invention are, for example, glycerol, lactic acid, pyrrolidone carboxylic acid and urea. In addition, it is particularly advantageous to use polymeric moisturizers from the group of water-soluble and/or water-swellable and/or water-gelable polysaccharides. Particularly advantageous are, for example, hyaluronic acid, chitosan and/or a fucose-rich polysaccharide which is listed in the Chemical Abstracts under the registry number 178463-23-5 and is available, for example, under the name Fucogel® 1000 from SOLABIA S. A.

The cosmetic and dermatological preparations according to the invention can comprise dyes and/or color pigments, particularly when they are in the form of decorative cosmetics. The dyes and color pigments can be chosen from the corresponding positive list of the Cosmetics Directive or the EC list of cosmetic colorants. In most cases they are identical to the dyes approved for foodstuffs. Advantageous color pigments are, for example, titanium dioxide, mica, iron oxides (e.g. Fe₂O₃, Fe₃O₄, FeO(OH)) and/or tin oxide. Advantageous dyes are, for example, carmine, Prussian blue, chromium oxide green, ultramarine blue and/or manganese violet. It is particularly advantageous to choose the dyes and/or color pigments from the Rowe Colour Index, 3rd edition, Society of Dyers and Colourists, Bradford, England, 1971.

If the formulations according to the invention are in the form of products which are used on the face, it is favorable to choose one or more substances from the following group: 2,4-dihdyroxyazobenzene, 1-(2′-chloro-4′-nitro-1′-phenylazo)-2-hydroxynaphthalene, Ceres red, 2-(sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 4-(4-sulfo-1-phenylazo)-1-(4-sulfophenyl)-5-hydroxypyrazolone-3-carboxylic acid. aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salts, aluminum salt of 2,4,5,7-tetraiodofluorescein, aluminum salt of quinophthalonedisulfonic acid, aluminum salt of indigodisulfonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extracts, β-carotene or cochineal.

Also advantageous for the purposes of the present invention are formulations with a content of pearlescent pigments. Preference is given in particular to the types of pearlescent pigments listed below:

1. Natural pearlescent pigments, such as, for example “pearl essence” (guanine/hypoxanthin mixed crystals from fish scales) and

“mother of pearl” (ground mussel shells)

2. Monocrystalline pearlescent pigments, such as, for example, bismuth oxychloride (BiOCl)

3. Layer-substrate pigments: e.g. mica/metal oxide

Bases for pearlescent pigments are, for example, pulverulent pigments or castor oil dispersions of bismuth oxychloride and/or titanium dioxide, and bismuth oxychloride and/or titanium dioxide on mica. The luster pigment listed under CIN 77163, for example, is particularly advantageous.

Also advantageous are, for example, the following types of pearlescent pigment based on mica/metal oxide:

| Group | Coating/layer thickness | Color |
|---|---|---|
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Prussian blue | deep blue |
| | TiO₂/carmine | red |

Particular preference is given, for example, to the pearlescent pigments obtainable from Merck under the trade names Timiron, Colorona and Dichrona.

The list of given pearlescent pigments is not of course intended to be limiting. Pearlescent pigments which are advantageous for the purposes of the present invention are obtainable by numerous methods known per se. For example, other substrates apart from mica can be coated with further metal oxides, such as, for example, silica and the like. SiO₂ particles coated with, for example, TiO₂ and Fe₂O₃ (“ronaspheres”), which are marketed by Merck and are particularly suitable for the optical reduction of fine lines are advantageous.

It may, moreover, be advantageous to dispense completely with a substrate such as mica. Particular preference is given to iron pearlescent pigments prepared without the use of mica. Such pigments are obtainable, for example, under the trade name Sicopearl Copper 1000 from BASF.

In addition, also particularly advantageous are effect pigments which are obtainable under the trade name Metasomes Standard/Glitter in various colors (yellow, red, green, blue) from Flora Tech. The glitter particles here are present in mixtures with various auxiliaries and dyes (such as, for example, the dyes with the Colour Index (CI) numbers 19140, 77007, 77289, 77491).

The dyes and pigments may be present either individually or in a mixture, and can be mutually coated with one another, different coating thicknesses generally giving rise to different color effects. The total amount of dyes and color-imparting pigments is advantageously chosen from the range from, for example, 0.1% by weight to 30% by weight, preferably from 0.5 to 15% by weight, in particular from 1.0 to 10% by weight, in each case based on the total weight of the preparations.

For the purposes of the present invention, it is also advantageous to provide cosmetic and dermatological preparations whose main purpose is not protection against sunlight, but which nevertheless have a content of UV protectants. Thus, for example, UV-A and/or UV-B filter substances are usually incorporated into day creams or make-up products. UV protectants, like antioxidants, and, if desired, preservatives, also constitute effective protection of the preparations themselves against spoilage. Also favorable are cosmetic and dermatological preparations in the form of a sunscreen.

Accordingly, for the purposes of the present invention, besides comprising one or more UV filter substances according to the invention, the preparations additionally comprise at least one further UV-A and/or UV-B filter substance. The formulations may, although not necessarily, optionally also comprise one or more organic and/or inorganic pigments as UV filter substances which may be present in the water and/or oil phase.

The preparations according to the invention can, in addition, also advantageously be in the form of so-called oil-free cosmetic or dermatological emulsions which comprise a water phase and at least one UV filter substance which is liquid at room temperature and/or one or more silicone derivatives as a further phase. Oil-free formulations for the purposes of the present invention can advantageously also comprise further lipophilic components—such as, for example, lipophilic active ingredients.

Particularly advantageous UV filter substances which are liquid at room temperature for the purposes of the present invention are homomenthyl salicylate (INCI: Homosalate), 2-ethylhexyl 2-cyano-3,3-diphenylacrylate (INCI: Octocrylene), 2-ethylhexyl 2-hydroxybenzoate (2-ethylhexyl salicylate, octyl salicylate, INCI: Octyl Salicylate) and esters of cinnamic acid, preferably 2-ethylhexyl 4-methoxycinnamate (INCI: Octyl Methoxycinnamate) and isopentyl 4-methoxycinnamate (INCI: Isoamyl p-Methoxycinnamate).

Preferred inorganic pigments are metal oxides and/or other metal compounds which are insoluble or sparingly soluble in water, in particular oxides of titanium (TiO₂), zinc (ZnO), iron (e.g. Fe₂O₃). zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and also the sulfate of barium (BaSO₄).

The pigments can, for the purposes of the present invention, also advantageously be used in the form of commercially available oily or aqueous predispersions. Dispersion auxiliaries and/or solubilization promoters may advantageously be added to these predispersions.

According to the invention, the pigments may advantageously by surface-treated (“coated”), the intention being to form or retain, for example, an amphiphilic or hydrophobic character. This surface treatment can consist in providing the pigments with a thin hydrophilic and/or hydrophobic inorganic and/or organic layer by processes known per se. The various surface coatings may also comprise water for the purposes of the present invention.

Inorganic surface coatings for the purposes of the present invention may consist of aluminum oxide (Al₂O₃), aluminum hydroxyide Al(OH)₃, and aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings can arise on their own or in combination and/or in combination with organic coating materials.

Organic surface coatings for the purposes of the present invention may consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may be present on their own, in combination and/or in combination with inorganic coating materials.

Zinc oxide particles suitable according to the invention and predispersions of zinc oxide particles are obtainable under the following trade names from the companies listed:

| Trade name | Coating | Manufacturer |
|---|---|---|
| Z - Cote HP1 | 2% dimethicone | BASF |
| Z - Cote | / | BASF |
| ZnO NDM | 5% dimethicone | H&R |

Suitable titanium dioxide particles and predispersions of titanium dioxide particles are obtainable under the following trade names from the companies listed:

| Trade name | Coating | Manufacturer |
|---|---|---|
| MT-100TV | aluminum hydroxide/stearic | Tayca |
| | acid | Corporation |
| MT-100Z | aluminum hydroxide/stearic | Tayca |
| | acid | Corporation |
| Eusolex T-2000 | alumina/simethicone | Merck KgaA |
| Titanium dioxide | octyltrimethylsilane | Degussa |
| T805 | | |
| (Uvinul TiO₂) | | |

An advantageous organic pigment for the purposes of the present invention is 2,2′-methylenebis(6-(2H-benzotriazol-2-yl)-4-(1,1,3,3-tetramethylbutyl)phenol) [INCI: Bisoctyltriazole], which is available under the trade name Tinosorb® M from CIBA-Chemikalien GmbH.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Advantageous further UV filter substances for the purposes of the present invention are sulfonated, water-soluble UV filters, such as, for example,

phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Bisimidazylate (CAS No.: 180898-37-7), which is available, for example, under the trade name Neo Heliopan AP from Haarmann & Reimer;

salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck or under Neo Heliopan Hydro from Haarmann & Reimer;

1,4-di(2-oxo-10-sulfo-3-bornylidenemethyl)benzene (also: 3,3′-(1,4-phenylene-dimethylene)bis(7,7-dimethyl-2-oxobicyclo[2.2.1]hept-1-ylmethane sulfonic acid) and salts thereof (particularly the corresponding 10-sulfato compounds, in particular the corresponding sodium, potassium or triethanolammonium salts), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)-sulfonic acid and salts thereof.

Advantageous UV filter substances for the purposes of the present invention are also so-called broadband filters, i.e. filter substances which absorb both UV-A and UV-B radiation.

Advantageous broadband filters or UV-B filter substances are, for example, triazine derivatives, such as, for example,

2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA Chemikalien GmbH;

dioctylbutylamidotriazone (INCI: Dioctylbutamidotriazone), which is available under the trade name UVASORB HEB from Sigma 3V;

tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine (INCI: Octyl Triazone), which is sold by BASF Aktiengesellschaft under the trade name UVINUL® T 150.

An advantageous broadband filter for the purposes of the present invention is 2,2′-methylenebis(6-(2H-benzotriazol-2-yl)₄-(1,1,3,3-tetramethylbutyl)phenol), which is available under the trade name Tinosorb® M from CIBA-Chemikalien GmbH.

A further advantageous broadband filter for the purposes of the present invention is 2-(2H-benzotriazol-2-yl)-4-methyl-6-[2-methyl-3-[1,3,3,3-tetramethyl-1-[(trimethylsilyl)-oxy]disiloxanyl]propyl]phenol (CAS No.: 155633-54-8) with the INCI name Drometrizole Trisiloxane.

The UV filter substances may be oil-soluble or water-soluble. Advantageous oil-soluble filter substances are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

2,4,6-trianilino(p-carbo-2′-ethyl-1′-hexyloxy)-1,3,5-triazine; esters of benzalmalonic acid, preferably di(2-ethylhexyl) 4-methoxybenzalmalonate;

esters of cinnamic acid, preferably 2-ethylhexyl 4-methoxycinnamate, isopentyl 4-methoxycinnamate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone and

UV filters bonded to polymers.

Advantageous water-soluble filter substances are, for example: sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

A further light protection filter substance to be used advantageously according to the invention is ethylhexyl 2-cyano-3,3-diphenylacrylate (octocrylene), which is obtainable from BASF under the name Uvinul® N 539.

The list of specified UV filters which may be used for the purposes of the present invention is not of course intended to be limiting.

Particularly advantageous preparations for the purposes of the present invention which are characterized by high or very high UV-A protection preferably comprise two or more UV-A and/or broadband filters, in particular dibenzoylmethane derivatives [for example 4-(tert-butyl)-4′-methoxydibenzoylmethane], benzotriazole derivatives [for example 2,2′-methylenebis(6-benzotriazol-2-yl)₄-(1,1,3,3-tetramethylbutyl)phenol)], phenylene-1,4-bis(2-benzimidazyl)-3,3′-5,5′-tetrasulfonic acid and/or its salts, 1,4-di(2-oxo-10-sulfo-3-bornylidenemethyl)benzene and/or salts thereof and/or 2,4-bis{[4-(2-ethylhexyloxy)-2-hydroxy]phenyl}-6-(4-methoxyphenyl)-1,3,5-triazine, in each case individually or in any combinations with one another.

The examples below serve to illustrate the present invention without limiting it. Unless stated otherwise, all amounts, fractions and percentages are based on the weight and the total amount or on the total weight of the preparations.

### EXAMPLES

### Example 1

### Foam-Like O/W Cream

| Emulsion I | % by wt. | % by vol. |
|---|---|---|
| Stearic acid | 5.00 | |
| Cetyl alcohol | 5.50 | |
| PEG-40 stearate | 8.50 | |
| Talc | 2.00 | |
| SiO₂ | 2.00 | |
| Magnesium aluminum silicate | 0.50 | |
| Paraffin oil | 5.00 | |
| Isohexadecane | 2.00 | |
| Glycerol | 5.00 | |
| Ceresin | 4.00 | |
| Sodium hydroxide | q.s. | |
| Preservative | q.s. | |
| Perfume | q.s. | |
| Water, demineralized | ad 100.00 | |
| pH adjusted to 6.5-7.5 | | |
| Emulsion I | | 70 |
| Nitrogen | | 30 |

Predispersion of the inorganic gel former and swelling of the hydrocolloid, and of the polymer with stirring in the water phase. Combining of the fatty phase heated to 75° C. with the water phase heated to 70° C. Addition of the particulate hydrophobic, hydrophobicized solid-body substances with stirring. Homogenization by means of a toothed-wheel dispersing machine (rotor-stator principle) at 65° C. 45 min stirring with gassing with nitrogen at 0.7 bar and cooling. Addition of the additives at 30° C. (perfume, active ingredients). Homogenization by means of a toothed-wheel dispersing machine (rotor-stator principle) at 27° C.

### Example 2

### Foam-Like O/W Lotion

| Emulsion II | % by wt. | % by vol. |
|---|---|---|
| Stearic acid | 4.00 | |
| Myristyl alcohol | 1.50 | |
| Cetylstearyl alcohol | 0.50 | |
| PEG-100 stearate | 4.00 | |
| Kaolin | 0.05 | |
| Hydroxyethylcellulose | 0.05 | |
| Magnesium aluminum silicate | 0.20 | |
| Mineral oil | 15.00 | |
| PEG-180/laureth-50/TMMG copolymer | 0.50 | |
| Glycerol | 3.00 | |
| Hydrogenated cocoa glyceride | 5.00 | |
| Sodium hydroxide | q.s. | |
| Preservative | q.s. | |
| Perfume | q.s. | |
| Water, demineralized | ad 100.00 | |
| pH adjusted to 5.0-6.5 | | |
| Emulsion II | | 50 |
| Gas (carbon dioxide) | | 50 |

Predispersion of the inorganic gel former and swelling of the hydrocolloid, and of the polymer with stirring in the water phase. Combining of the fatty phase heated to 80° C. with the water phase heated to 72° C. Addition of the particulate hydrophobic, hydrophobicized solid-body substances with stirring. Homogenization by means of a toothed-wheel dispersing machine (rotor-stator principle) at 65° C. 45 min stirring with gassing with carbon dioxide at 1.2 bar and cooling. Addition of the additives at 30° C. (perfume). Homogenization by means of a toothed-wheel dispersing machine (rotor-stator principle) at 30° C.

### Example 3

### Foam-Like O/W Lotion

| Emulsion III | % by wt. | % by vol. |
|---|---|---|
| Stearic acid | 2.00 | |
| Cetylstearyl alcohol | 2.50 | |
| PEG-30 stearate | 3.00 | |
| Aluminum starch octenylsuccinate | 3.00 | |
| Talc | 0.50 | |
| Polyurethane | 0.10 | |
| Magnesium silicate | 0.10 | |
| PEG-180/octoxynol-40/TMMG copolymer | 0.25 | |
| Dimethicones | 1.00 | |
| Cetyl palmitate | 1.00 | |
| Cera microcristillina | 1.00 | |
| Polydecene | 10.00 | |
| Citric acid | 0.10 | |
| Glycerol | 3.00 | |
| Perfume, preservative | q.s. | |
| Sodium hydroxide | q.s. | |
| Dyes etc. | q.s. | |
| Water | ad 100.00 | |
| pH adjusted to 6.0-7.5 | | |
| Emulsion III | | 65 |
| Gas (air) | | 35 |

Predispersion of the inorganic gel former and swelling of the hydrocolloids, and of the polymer with stirring in the water phase. Combining of the fatty phase heated to 80° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in an open reactor up to 30° C. Addition of the additives at 30° C. (perfume, active ingredients). Homogenization by means of a toothed-wheel dispersing machine (rotor-stator principle) at 25° C.

### Example 4

### Foam-Like O/W Emulsion Make-Up

| Emulsion IV | % by wt. | % by vol. |
|---|---|---|
| Palmitic acid | 3.00 | |
| Cetyl alcohol | 3.00 | |
| PEG-100 stearate | 3.00 | |
| Polyacrylic acid | 0.10 | |
| Wheat starch | 2.00 | |
| Kaolin | 4.50 | |
| Sodium magnesium silicate | 0.15 | |
| Dimethicone | 0.50 | |
| Hydrogenated polyisobutene | 9.50 | |
| Dicaprylyl ether | 2.00 | |
| Cetyl ricinoleate | 2.00 | |
| Glycerol | 3.00 | |
| Mica | 1.00 | |
| Iron oxides | 1.00 | |
| Titanium dioxide | 4.50 | |
| Vitamin A palmitate | 0.10 | |
| Sodium hydroxide | q.s. | |
| Preservative | q.s. | |
| Perfume | q.s. | |
| Water demineralized | ad 100.00 | |
| pH adjusted to 6.0-7.5 | | |
| Emulsion IV | | 37 |
| Gas (oxygen) | | 63 |

Predispersion of the inorganic gel formers and swelling of the hydrocolloid, and of the polymer with stirring in the water phase. Combining of the fatty and pigment phase heated to 78° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with oxygen at 1.3 bar with cooling to 30° C. Addition of the aluminum starch octenylsuccinate, the manioc starch, the perfume and the active ingredients at 30° C. Homogenization by means of a toothed-wheel dispersing machine (rotor-stator principle) at 25° C.

### Example 5

### Foam-Like O/W Cream

| Emulsion V | % by wt. | % by vol. |
|---|---|---|
| Stearic acid | 2.00 | |
| Cetyl alcohol | 1.00 | |
| PEG-30 stearate | 1.00 | |
| Isohexadecanes | 10.00 | |
| Cyclomethicone | 1.00 | |
| Shea butter | 2.50 | |
| Ozokerite | 0.50 | |
| Vitamin E acetate | 1.00 | |
| Retinyl palmitate | 0.20 | |
| Glycerol | 3.00 | |
| BHT | 0.02 | |
| Na₂H₂EDTA | 0.10 | |
| Carboxymethylcellulose | 0.05 | |
| Quaternium-18 hectorite | 0.20 | |
| Perfume, preservative | q.s. | |
| Dyes | q.s. | |
| Potassium hydroxide | q.s. | |
| Water | ad 100.00 | |
| pH adjusted to 5.0-7.0 | | |
| Emulsion V | | 43 |
| Gas (nitrous oxide) | | 57 |

Predispersion of the inorganic gel formers and swelling of the hydrocolloid, and of the polymer with stirring in the water phase. Combining of the fatty phase heated to 80° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with nitrous oxide at 0.7 bar with cooling to 30° C. Addition of the additives at 30° C. (perfume, active ingredients). Homogenization by means of a toothed-wheel dispersing machine (rotor-stator principle) at 26° C.

### Example 6

### Foam-Like O/W Lotion

| Emulsion VI | % by wt. | % by vol. |
|---|---|---|
| Stearic acid | 3.00 | |
| Cetylstearyl alcohol | 2.00 | |
| PEG-100 stearate | 2.00 | |
| Paraffin oil | 6.50 | |
| Dimethicone | 0.50 | |
| Vitamin E acetate | 2.00 | |
| Vaseline (petrolatum) | 3.50 | |
| Glycerol | 3.00 | |
| Carboxymethylcellulose | 0.05 | |
| Magnesium aluminum silicate | 0.50 | |
| Kaolin | 0.05 | |
| Talc | 0.50 | |
| PEG-180/laureth-50/TMMG copolymer | 0.50 | |
| Perfume, preservative | q.s. | |
| Dyes etc. | q.s. | |
| Sodium hydroxide | q.s. | |
| Water | ad 100.00 | |
| pH adjusted to 6.0-7.5 | | |
| Emulsion VI | | 35 |
| Gas (argon) | | 65 |

Predispersion of the inorganic gel formers and swelling of the hydrocolloid, and of the polymer with stirring in the water phase. Combining of the fatty phase heated to 78° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with argon at 1 bar with cooling to 30° C. Addition of the additives at 30° C. (perfume, active ingredients). Homogenization by means of a toothed-wheel dispersing machine (rotor-stator principle) at 23° C.

### Example 7

### Foam-Like Sunscreen Cream

| Emulsion VII | % by wt. | % by vol. |
|---|---|---|
| Stearic acid | 1.00 | |
| Cetylstearyl alchol | 2.00 | |
| PEG-20 stearate | 1.00 | |
| Caprylic/capric triglycerides | 2.00 | |
| Paraffin oil | 15.50 | |
| Dimethicone | 0.50 | |
| Octyl isostearate | 5.00 | |
| Myristyl myristate | 2.00 | |
| Glycerol | 3.00 | |
| Octyl methoxycinnamate | 4.00 | |
| Butylmethoxydibenzoylmethane | 3.00 | |
| Ethylhexyltriazone | 3.00 | |
| BHT | 0.02 | |
| Na₂H₂EDTA | 0.10 | |
| Perfume, preservative | q.s. | |
| Dyes, etc. | q.s. | |
| Potassium hydroxide | q.s. | |
| Water | ad 100.00 | |
| pH adjusted to 5.0-6.0 | | |
| Emulsion VII | | 35 |
| Gas (helium) | | 65 |

Combining of the fatty/photoprotection filter phase heated to 78° C. with the water/photoprotection filter phase heated to 75° C. Homogenization by means of a toothed-wheel dispersing machine (rotor-stator principle) at 65° C. 45 min stirring in a Becomix with gassing with helium at 1 bar with cooling to 30° C. Addition of the additives at 30° C. (perfume). Homogenization by means of a toothed-wheel dispersing machine (rotor-stator principle) at 23° C.

That Which is claimed:

1. A self-foaming, foam-like, after-foaming or foamable cosmetic or dermatological preparation comprising at least one compound that is solid or semi-solid at room temperature selected from the group consisting of waxes and lipids.

2. The preparation as claimed in claim 1, wherein the at least one compound is present in an amount from 0.05 to 10% by weight, based on the total weight of the preparation.

3. The preparation as claimed in claim 1, wherein the at least one compound is present in an amount from 0.1 to 5% by weight, based on the total weight of the preparation.

4. The preparation as claimed in claim 1, further comprising one or more gases in an amount from 10 to 80% by volume, based on the total volume of the preparation.

5. The preparation as claimed in claim 1, wherein the preparation includes carbon dioxide.

6. The preparation as claimed in claim 1, further comprising one or more moisturizers.

7. The preparation as claimed in claim 1, wherein the at least one compound includes at least one vegetable wax.

8. The preparation as claimed in claim 7, wherein the vegetable wax is selected from the group consisting of shea butter and cocoa butter.

9. The preparation as claimed in claim 1, wherein the at least one compound includes at least one animal wax.

10. The preparation as claimed in claim 1, wherein the at least one compound includes at least one mineral wax.

11. The preparation as claimed in claim 1, wherein the at least one compound includes at least one chemically modified or synthetic wax.

12. The preparation as claimed in claim 1, wherein the at least one compound includes at least one ester wax, glycol ester or glyceride.

13. The preparation as claimed in claim 1, wherein the at least one compound includes at least one paraffin wax.

14. The preparation as claimed in claim 1, wherein the at least one compound includes at least one lipid selected from the group consisting of solid and semi-solid paraffin hydrocarbons.

15. The preparation as claimed in claim 1, wherein the at least one compound includes at least one lipid selected from the group consisting of synthetic and semi-synthetic lipids.

16. A skincare or hair care method, comprising applying to the skin or hair a self-foaming, foam-like, after-foaming or foamable cosmetic or dermatological preparation comprising at least one compound that is solid or semi-solid at room temperature selected from the group consisting of waxes and lipids.

17. The method as claimed in claim 16, wherein the at least one compound is present in an amount from 0.05 to 10% by weight, based on the total weight of the preparation.

18. The method as claimed in claim 16, wherein the at least one compound is present in an amount from 0.1 to 5% by weight, based on the total weight of the preparation.

19. The method as claimed in claim 16, wherein the at least one compound includes at least one vegetable wax.

20. The method as claimed in claim 19, wherein the vegetable wax is selected from the group consisting of shea butter and cocoa butter.

21. The method as claimed in claim 16, wherein the at least one compound includes at least one lipid selected from the group consisting of solid and semi-solid paraffin hydrocarbons.

22. The method as claimed in claim 16, wherein the at least one compound includes at least one lipid selected from the group consisting of synthetic and semi-synthetic lipids.

23. A method for boosting the foam of self-foaming, foam-like, after-foaming or foamable cosmetic or dermatological preparation comprising the step of adding to the preparation at least one compound that is solid or semi-solid at room temperature selected from the group consisting of waxes and lipids.

24. The method as claimed in claim 23, wherein the at least one compound is present in an amount from 0.05 to 10% by weight, based on the total weight of the preparation.

25. The method as claimed in claim 23, wherein the at least one compound is present in an amount from 0.1 to 5% by weight, based on the total weight of the preparation.

26. The method as claimed in claim 23, wherein the at least one compound includes at least one vegetable wax.

27. The method as claimed in claim 26, wherein the vegetable wax is selected from the group consisting of shea butter and cocoa butter.

28. The method as claimed in claim 23, wherein the at least one compound includes at least one lipid selected from the group consisting of solid and semi-solid paraffin hydrocarbons.

29. The method as claimed in claim 23, wherein the at least one compound includes at least one lipid selected from the group consisting of synthetic and semi-synthetic lipids.
